(19) United States
(12) Patent Application Publication
(10) Pub. No.: US 2011/0190482 A1
(43) Pub. Date: Aug. 4, 2011

(54) POLYMER ENCAPSULATED ALUMINUM PARTICULATES

(75) Inventors: Villoo Morawala Patell, Bangalore (IN); Rajesh Ullanat, Bangalore (IN); Thippeswamy Sidegonde, Bangalore (IN); Sunil Shekar, Bangalore (IN); Sunit Maity, Bangalore (IN); Chellappa Gopalakrishnan, Bangalore (IN); Sami Noshir Guzder, Bangalore (IN)

(73) Assignee: AVESTHAGEN LIMITED, Bangalore, Karnataka (IN)

(21) Appl. No.: 12/997,215

(22) PCT Filed: Jun. 10, 2009
(86) PCT No.: PCT/IB2009/005899

§ 371 Date: Dec. 9, 2010

(30) Foreign Application Priority Data
Jun. 10, 2008 (IN) 01411/CHE/2008

(51) Int. Cl. C07H 21/00; C40B 20/00

(52) U.S. Cl. 536/23.1; 506/2

(57) ABSTRACT

The present invention relates to use of novel bioinformatics approach for predicting and identifying Scaffold/Matrix attachment regions (S/MARs) from different genomic database.

### FIELD OF THE INVENTION

The present invention relates to use of novel bioinformatics approach for predicting and identifying Scaffold/Matrix attachment regions (S/MARs) from different genomic database.

### BACKGROUND AND PRIOR ART OF THE INVENTION

A variety of patterns have been observed on the DNA sequences and proteins that serve as control points for gene expression and cellular functions. Owing to the vital role of such patterns, these patterns are of great interest. Among these S/MARs (Scaffold/Matrix attachment regions, abbreviated as S/MARs) is one of the most important DNA sequences. In the nucleus of eukaryotic cells specific regions of the DNA are attached to the nuclear matrix. These regions are called S/MARs. It is believed that there are tens of thousands of S/MARs in the genome of higher organisms (Boulikas, T. 1995). They are believed to be responsible for attachment of chromatin loops to the nuclear scaffold or matrix Meng et al. 2004). These sequences are involved in chromatin remodeling and subsequent transcriptional activation and also protection of transgenes from position effect (Widak, W. and Widlak, P. 2004, Cockerill et al. 1987 and Walter et al. 1998). They also have a strong effect on the level of expression of transgenes as shown by Allen, G C. et al. in 2000. Insertion of these sequences into the vector backbone has been shown to enhance the expression of therapeutics proteins (Girod, P A. and Mermod, N. 2003).

One of the major constraints with experimental detection of S/MARs is that it exhibits variation in length and nucleotide sequence, this trait is yet to be explored. So experimental detection is not suitable for large-scale screening of genomic sequences and thus bioinformatics approach is a prerequisite for the analysis of whole genomes.

Several bioinformatics methods of S/MAR prediction have been developed as a result of considerable amount of research. The MAR-Finder method scores sub-sequences of DNA by the abundance of DNA-motifs thought to be correlated with S/MARs (Singh et al. 1997). SMARTest (Frisch et al. 2002) and ChrClass (Glazko et al. 2001) are two different methods which used a training set in predicting motifs. Basis of Mar-Wiz rule in predicting S/MAR is that a long run of bases that do not contain a G binds to the matrix (Dickinson et al. 1992). Kieffer et al. calculated free energy to predict S/MARs(Thermodyn). In addition, experimental groups have suggested particular motifs: the MAR recognition signature (MRS) consisting of two consensus sequences (van Drunen et al. 1999) and a “consensus” sequence by Wang et al. in 1995. Recently researchers at Selexis SA and The University of Lausanne have reported identification of MARs using a novel bioinformatics approach, called SMARScan (Girod et al. 2007), which suggests that S/MAR sequences adopt a curved DNA structure and binds specific transcription factors.

MAR-Finder

The MAR-Finder method utilizes the pattern-density on DNA sequence as the basis for predicting the occurrence of Matrix Association Regions or MARs. It uses a set of DNA-sequence motifs that have been biologically known to be present in S/MARs. In a window of fixed length the number of occurrences of each motif is determined and compared to the expected number of occurrences in a random DNA sequence of the same length as the window. Using statistical algorithm MAR-potential is calculated which is average of the score for both positive and negative strand. This step is repeated for each window along the sequence and those windows that have a MAR-potential above a given threshold are predicted to contain a putative S/MAR.MAR-Finder gives a sensitivity of 32% and a precision of 80%.

MAR-Wiz Rule

It has been found that a long run of bases that do not contain a G binds to the matrix [14]. Computational approach to find MARs in MAR-Wiz is based upon the co-occurrence of 20 DNA patterns that have been known to occur in the neighborhood of MARs. These motifs are used to define higher order rules that are in-turn defined using the various combinations in which the patterns have been known to co-occur. The mathematical density of the rule occurrences in a region is assumed to imply the presence of a MAR in that region.

MRS Signature

MAR recognition signature, is a bipartite sequence that consists of two individual sequences AATAAYAA and AWWRTAANNWWGNNNC. It has been suggested to be an indicator for the presence of S/MAR, where Y=C or T, W=A or T, R=A or G, and N=A or C or G or T. It has been suggested that these motifs should appear within about 200 bp of each other independent of strand and order and could even be overlapping.

SMARTest

This approach is based on a library of S/MAR-associated, AT-rich patterns derived from comparative sequence analysis of experimentally defined S/MAR sequences. Initially by using experimentally defined S/MAR sequences as the training set and a library of new S/MAR-associated, AT-rich patterns described as weight matrices was generated. Then performing a density analysis based on the S/MAR matrix library, potential S/MARs were identified. Currently, proprietary library of 97 S/MAR-associated weight matrices are used to test genomic DNA sequences for the occurrence of potential regions of S/MARs. S/MAR predictions were also evaluated by using six genomic sequences from animal and plant for which S/MARs and non-S/MARs were experimentally mapped. SMARTest reached a sensitivity of 38% and a specificity of 68%.

SMARScan

SMARScan works on the hypothesis, which involves activation of gene expression by MARs, which may require sequences determining structural properties of the DNA, such as DNA curvature, as well as motifs serving as binding sites for transcription factors. The SMARScan I program was assembled to automatically compute structural features of DNA using the GeneExpress algorithms designed to predict the melting temperature, curvature, major grove depth and minor grove width of the DNA and later SMARScan I was coupled to the prediction of potential transcription factor binding sites, resulting in SMARScan II.

ChrClass

Multivariate linear discriminant analysis revealed significant differences between frequencies of simple nucleotide motifs in S/MAR sequences and in sequences extracted directly from various nuclear matrix elements, such as nuclear lamina, cores of rosette-like structures, synaptonemal complex. Based on this result ChrClass was developed for the prediction of the regions associated with various elements of the nuclear matrix in a query sequence.

Stress-Induced Destabilization

Stress-induced destabilization (SIDD) calculations predict where the DNA strands can easily separate: it has been suggested that this is an indication of the presence of an S/MAR (Benham et al. 1997). It has been shown by computational analysis that S/MARs conform to a specific design whose essential attribute is the presence of stress-induced base-unpairing regions (BURs). SIDD profiles are calculated later using a previously developed statistical mechanical procedure in which the superhelical deformation is partitioned between strand separation, twisting within denatured regions, and residual superhelicity.

Consensus Sequence

The consensus sequence consisted of concatemerized repeats of a 25-base pair SATB1 recognition sequence (TCTTTAATTTCTAATATATTTAGAA), which is derived from the core unwinding element of the MAR downstream of the mouse immunoglobulin heavy chain enhancer.

Thermodyn

Thermodyn is a calculation of the free energy of strand separation derived from summing the contributions of each doublet in a window to the thermodynamic quantities ΔH and ΔS.

AT-Percentage

A simple measure of AT-percentage was also used for predicting S/MARs. AT percentage was calculated as the proportion of bases that are A or T in a sliding window of 300 bases.

Comparing studies between different methods (Evans et al. 2007) has suggested that that existing methods can definitely pick out few really true positive S/MARs, however, it is also clear that there is a need of a new bioinformatics approach, which will identify S/MARs with good precision. In contrast to previous algorithms developed for prediction of S/MARs that were based on pattern and density analysis, a new approach based on gene expression levels has been developed. In this study, a genome scale analysis of expression level to predict the intergenic S/MAR elements has been undertaken. Experimentally defined S/MAR sequences were used as the training set and a library of new S/MAR-associated sequences has been generated based on higher and constitutive gene expression. This approach is independent of sequence context and is suitable for the analysis of complete chromosomes. These findings will open new perspectives for the identification of S/MARs, which will help in understanding the importance of S/MARs in gene regulation.

Considerations for Vector Design Using S/MAR Sequence

A. The Length of the Loop

While it is generally agreed that the average size of a chromatin domain in a eukaryotic cell is around 70 kb, the natural distribution of S/MARs reveals sizes ranging between 3 and about 200 kb (Gasser and Laemmli, 1987). Generally the smaller loop sizes are assigned to genes that can be highly transcribed under certain circumstances and prototype examples for this may be the histone gene cluster (5 kb) which is regulated in a cell-cycle dependent fashion and the type I interferon gene cluster (loop sizes 3-14 kb; Strissel et al., 1998) members of which are rapidly activated following a viral infection. It is proposed that these loci are permanently potentiated as a possible consequence of the close apposition of S/MARs. (Bode et al., 2000)

B. Placement of S/MARS Both 5′ and 3′ of the Gene

S/MARs repeated over a short distance might sterically interfere with a cooperative 10 to 30 nm fiber transition and thereby counteract inactivation. In accord with such a model an artificial S/MAR-luciferase-S/MAR minidomain with a 3 kb loop was found to remain active after transfection for more than 3 month whereas a truncated control (S/MAR-luciferase) construct, for which the loop size is determined by the genomic site of integration, lost half its expression over a period of 6 weeks (Bode et al., 1995). In contrast to these small, permanently open domains, genes that are only expressed in distinct cell types or at certain stages of development are typically embedded in larger domains which have to acquire transcriptional competence under the respective circumstances (Bode et al., 2000).

C. Retrovirus Binds to DNA Regions with High Transcription-Promoting Potential

The eukaryotic genome contains chromosomal loci with a high transcription-promoting potential. For their identification in cultured cells, transfer of a reporter gene has to be performed by a technique that grants the integration of individual copies. We have applied retroviral vectors in conjunction with inverse polymerase chain reaction techniques to reconstruct a number of these sites for a further characterization. Remarkably, all examples conform to the same design in that the process of retroviral infection selected a scaffold- or matrix-attached region (S/MAR) that was flanked by DNA with high bending potential. The S/MARs are of an unusual type in that they show a high incidence of certain dinucleotide repeats and the potential to act as topological sinks. The anatomy of retroviral integration sites reveals principles that can be exploited for the development of predictable transgenic systems on the basis of expression and targeting vectors. (Schübeler D et al., 1996)

D. Definition of the Distance Between the S/MAR and the Transcriptional Start Site (TSS)

Scaffold/matrix-attached regions (S/MARs) are cis-acting elements with a function outside transcribed regions and in introns. Although they usually augment transcriptional rates, their action is highly context-dependent. We cloned an 800 bp S/MAR element from the upstream border of the human interferon-beta domain at various positions within a transcribed region of 4.3 kb. By use of retroviral gene transfer, the vector could be integrated into target cells as a single copy enabling a rigorous definition of the distance between the S/MAR and the transcriptional start site. At a distance of about 4 kb, the S/MAR supported transcriptional initiation, whereas at distances below 2.5 kb, transcription was essentially shut off. Controls proved the functionally of all constructs in the transient expression phase and ruled out any influence of S/MAR position on transcript stability. Moreover, no pausing or premature termination was observed within these elements. We suggest that the protein binding partners of S/MARs change according to the topological status, explaining these divergent S/MAR effects. (Schübeler D et al., 1996)

Databases Used

A. Ensembl

Ensembl database was used to extract information regarding gene coordinates, chromosome number, and strand, for all the genes in our dataset obtained from H-Inv database. Ensembl database version 48 was used.

B. UniGene

UniGene is an organized View of the transcriptome. Each UniGene entry is a set of transcript sequences that appear to come from the same transcription locus (gene or expressed pseudogene), together with information on protein similarities, gene expression, cDNA clone reagents, and genomic location. UniGene Build #216 was used.

### REFERENCES

1. Boulikas, T. Int Rev Cytol. 162A, 279-388 (1995)
2. Heng, H H Q. et al. J Cell Sci. 117, 999-1008 (2004)
3. Widak, W. and Widlak, P. Cell Mol Biol Lett. 9, 123-133 (2004)
4. Cockerill, P N. et al. J Biol Chem. 262, 5394-5397 (1987)
5. Walter, W R. et al. Biochem Biophys Res Commun. 242, 419-422 (1998)
6. Allen, G C. et al. Plant Molecular Biology. 43, 361-176 (2000)
7. Girod, P A. and Mermod, N. Gene Transfer and Expression in Mammalian Cells, Elsevier Sciences, 359-379 (2003)
8. Singh, GB. et al. NAR. 25, 1419-1425 (1997)
9. Frish, M. et al. Genom. Biol. 12, 349-354 (2002)
10. Glazko, G V. et al. Biochim Biophys Acta. 1517, 351-364 (2001)
11. Dickinson, L A. et al. Cell. 70, 631-645 (1992)
12. van Drunnen, C M. et al. NAR. 27, 2924-2930 (1999)
13. Wang, B. et al. J Biol Chem. 270, 23239-23242 (1995)
14. Girod, P A. et al. Nature Mehtods. 4, 747-753 (2007)
15. Benham, C. et al. J Mol Biol. 274, 181-196 (1997)
16. Evans, K. et al. BMC Bioinformatics. 8, 71-99 (2007)
17. Bode et al., Crit Rev Eukaryot Gene Expr.; 10(1): 73-90 (2000)
18. Schübeler D et al., Biochemistry. 35(34): 11160-9 (1996)

### OBJECTS OF THE INVENTION

The main object of the present invention is to develop a method for identifying Scaffold/Matrix attachment region(S/MAR) sequence.

Another object of the present invention is to obtain a Scaffold/Matrix attachment region (S/MAR) sequence[s] or its complementary sequence[s], variant[s] and fragment[s] thereof.

Yet another object of the present invention is to use (S/MAR) sequence[s] or its complementary sequence[s], variant[s] and fragment[s] for increased protein production through enhanced expression of genes.

### SUMMARY OF THE INVENTION

The present invention relates to a method for identifying Scaffold/Matrix attachment region(S/MAR) sequence, said method comprising steps of (a) generating a library of subset of genes based on higher and constitutive gene expression predicted from datasets derived from human autonomic gene expression library; and (b) assessing 5′ UTR intergenic sequences for the subsets to identify the MAR sequence; and a Scaffold/Matrix attachment region (S/MAR) sequence[s] or its complementary sequence[s], variant[s] and fragment[s] thereof.

### DESCRIPTION OF FIGURES

FIG. 1: Determining enrichment of S/MAR motifs in known S/MAR sequences

FIG. 2: Identifying S/MAR sequences

FIG. 3: S/MAR Workflow.

FIG. 4: Count of S/MAR motifs/160 KB for S/MARt DB seq, intergenic upstream of constitutive & low exp. genes and exons

FIG. 5: S/MAR motif counts in intergenic region of constitutively expressed genes by seq length

FIG. 6: S/MAR motif counts in intergenic region upstream of low expressing genes by seq length

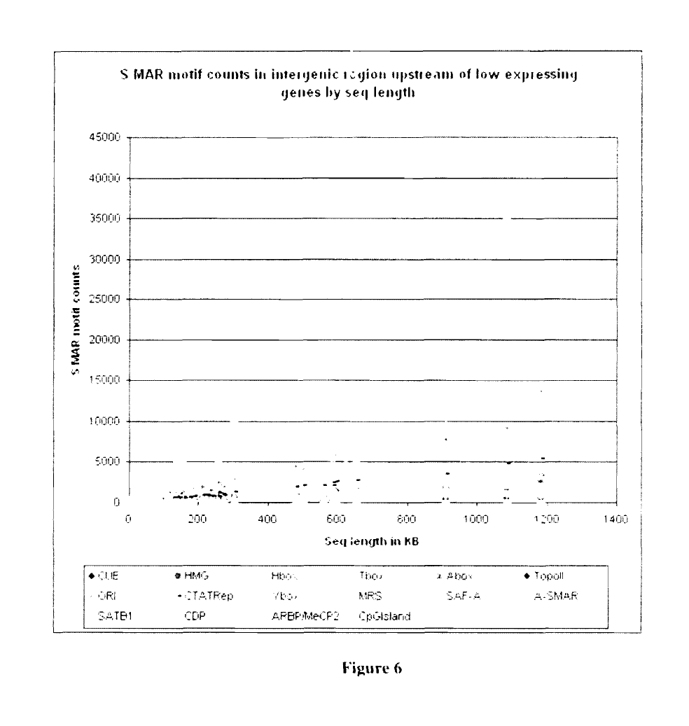

FIG. 7: S/MAR motif counts in intergenic region containing the S/MARt DB seq per KB

FIG. 8: S/MAR motif counts/KB in constitutively expressed genes

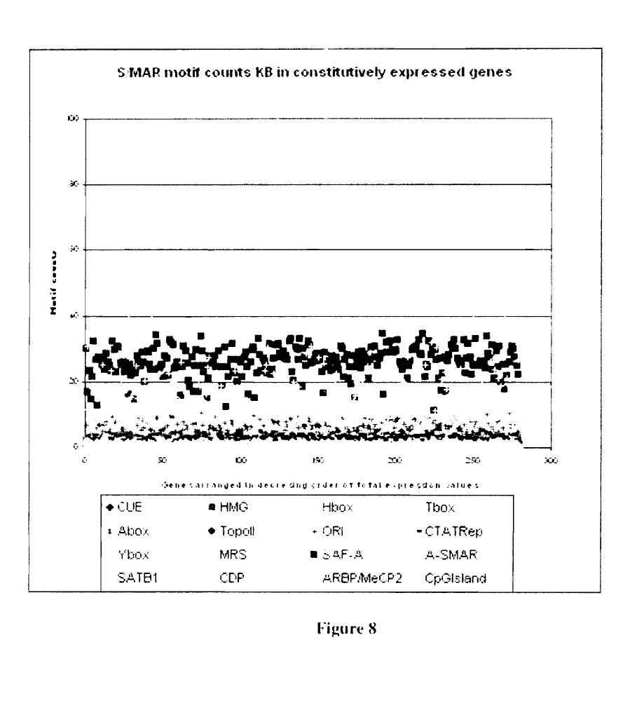

FIG. 9: S/MAR motif counts/KB in constitutively expressed genes

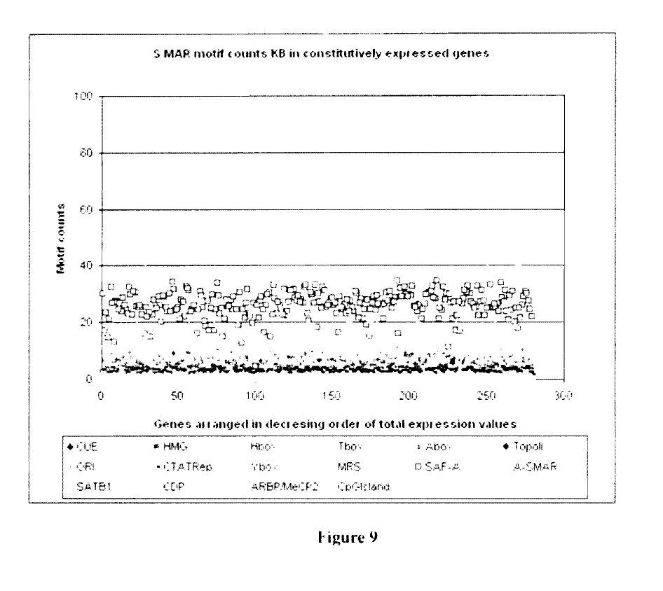

FIG. 10: S/MAR motif counts/KB for low expressing genes

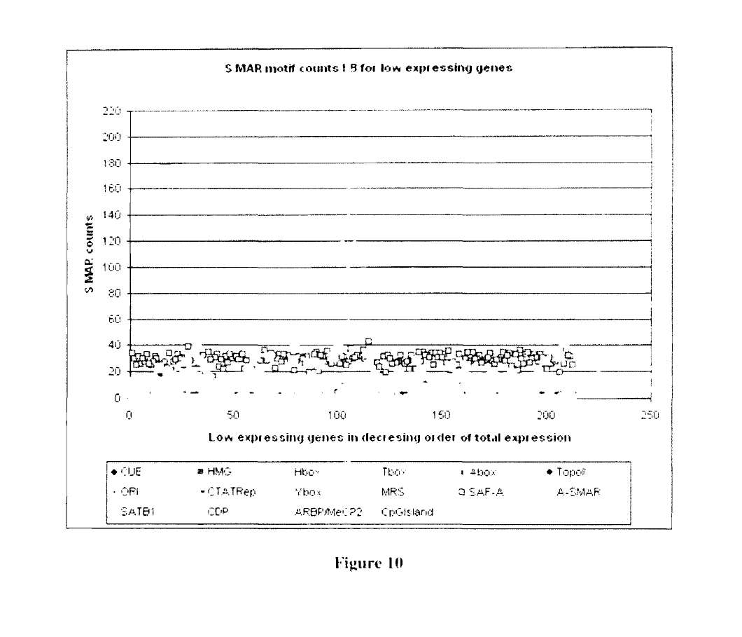

### DETAILED DESCRIPTION OF THE INVENTION

Scaffold/matrix attachment regions (S/MARs) are operationally defined as DNA elements that bind specifically to the nuclear matrix or as DNA fragments that co purify with the nuclear matrix. S/MARs are sequences in the DNA of eukaryotic chromosomes where the nuclear matrix attaches. These elements constitute anchor points of the DNA for the chromatin scaffold and serve to organize the chromatin into structural domains. These are found at the base of the chromatin loops into which the eukaryotic genome appears to be organized.

These regions are about 300 bp to several kb in length and are present in all higher eukaryotes, including mammals and plants (Bode et al., 1996; Allen et al., 2000). S/MARs are notable for their AT richness and likely narrowing of the minor groove (Gasser et al., 1989; Bode et al., 1995, 1996). They belong to non coding sites in the genome. Scaffold/matrix attachment regions (S/MARs) are essential regulatory DNA elements of eukaryotic cells.

Functionally MARs are very important as they participate in many cellular processes. They typically augment transcription rates in a highly context dependent manner (Schubeler et al., 1996) but are separable from enhancer sequences on the basis of transient expression analyses (Bode et al., 1995). S/MAR act independent of orientation and independent of distance, provided it is at least several kilo bases. They can activate enhancer regions (Cockerill et al., 1987) and determine which one of a class of genes to transcribe (Walter et al., 1998). They also have a strong effect on the level of expression of transgenes (Allen et al., 2000; Girod et al., 2005).

The promoter-S/MAR distance is an important factor in the correct functioning of the S/MAR. (Mlynarova et al., 1995; Schubeler et al., 1996). In addition to the S/MAR-associated enhancement of gene expression, S/MARs have a proposed role in the negative regulation of gene expression. Such negative regulation is the proposed default mode of action for S/MARs both closely associated with the promoter sequence or when appearing downstream of the promoter (Schubeler et al., 1996). Such S/MARs would block progression by RNA polymerase II, so they may be either nonfunctional in vivo or have a regulated matrix-binding activity (Schubeler et al., 1996).

An additional feature of MARs is their function as origins of replication in combination with other genetic elements. MAR AT-rich sequences were reported to facilitate dissociation of the two DNA strands, and may thereby open chromatin and allow interaction with factors of the DNA replication machinery. This has allowed the construction of episomally replicating expression vectors for mammalian cells. Due to these features of S/MAR, they are of intrinsic interest for the understanding of gene regulation, which will help to enhance gene expression and increased protein production in eukaryotic cells. But MARs exhibits lots of variations in length and nucleotide sequence, which is still unexplored and so experimental detection is not suitable for large-scale screening of genomic sequences. Hence bioinformatics approach is a prerequisite for the analysis of whole genomes.

A great deal of research work has been focused on computer prediction of S/MARs. A number of methods have been proposed to predict S/MAR as MAR-finder (Singh et al., 1997), H rule (Dickinson et al., 1992), MRS signature, SMARtest (Frisch et al., 2002), Duplex Destabilization and Thermodyne etc. Evans et al compared them. And from their study they concluded that all the methods have little predictive power and a simple rule based on A-T percentage is generally competitive with other methods (Evans et al, 2007)

In this project, we are concentrating on “in silico Prediction of Human Scaffold/Matrix Attachment Regions specifically enhancing gene expression”. Expression data and sequence information were obtained from UniGene and Ensembl respectively. The sequences will be screened for specific S/MAR features and potential candidate sequences will be identified by in-house algorithm. The identified S/MAR sequences will be used for construction of episomally replicating high expression vectors for mammalian cells (Table 1).

TABLE 1
Patterns and motifs for identification of S/MAR sequences

| | | | Short |
|---|---|---|---|
| Motif name | Pattern | References | name |
| Core unwinding | ATATTT/ATATAT/AATATATTT/ | 2, 3, 4 | CUE |
| motifs (CUEs) | AATATATTAATATT | | |
| HMG-I/Y protein | TATTATATAA/TAATAAAATTTT | 2, 37 | HMG |
| binding sites | | | |
| H-box (A/T25) | [ATC]{25,} | 5 | Hbox |
| T-Box | TT[AT]T[AT]TT[AT]TT | 3, 2 | Tbox |
| A-Box | AATAAA[TC]AAA | 3, 2 | Abox |
| Topoisomerase II | [AG][ATGC][TC][ATGC][ATGC] | 2, 3, 6 | TopoII |
| binding sites | C[ATGC][ATGC]G[TC][ATGC] | | |
| | G[GT]T[ATGC][TC][ATGC][TC]/ | | |
| | GT[ATGC][AT]A[CT]ATT[ATGC] | | |
| | AT[ATGC][ATGC][AG] (Missed | | |
| | the starting ‘GTN’ for | | |
| | Drosophila. Have added here) | | |
| Origin of | ATTA/ATTTA | 1, 2 | ORI |
| replication | | | |
| CTAT repeats-binding | CTAT | 2 | CTATRep |
| proteins regions | | | |
| Y-box | CCAAT | 2 | Ybox |
| MAR recognition | AATAA[TC]AA and A[AT][AT] | 2 | MRS |
| signature | [AG]TAA[ATGC][ATGC][AT] | | |
| | [AT]G[ATGC][ATGC][ATGC]C | | |
| | within 200 bP | | |
| SAF-A binding region | A{3,}\|T{3,} | 9 | SAF-A |
| [A{3,}/T{3,}pattern] | | | |
| Arabidopsis S/MARs | TA[AT]A[AT][AT][AT][ATGC] | 6 | A-SMAR |
| | [ATGC]A[AT][AT][AG]TAA | | |
| | [ATGC][ATGC][AT][AT]G | | |
| SATB1 binding site | TATTA[GCA]{1,2}TAATAA/ | 10 | SATB1 |
| | AA[TA]TTCTAATAT | | |
| CDP binding sites | AT[CT]GAT[TCA]A[ATGC][T/C]/ | 11, 12, 13 | CDP |
| | [CT]GAT[TCA]A[ATGC][TC] | | |
| CpG islands. | Use EMBOSS CpGplot | 2 | CpGIsland |
| ARBP/MeCP2 binding | GGTGT | 14, 15 | ARBP/ |
| regions | | | MeCP2 |

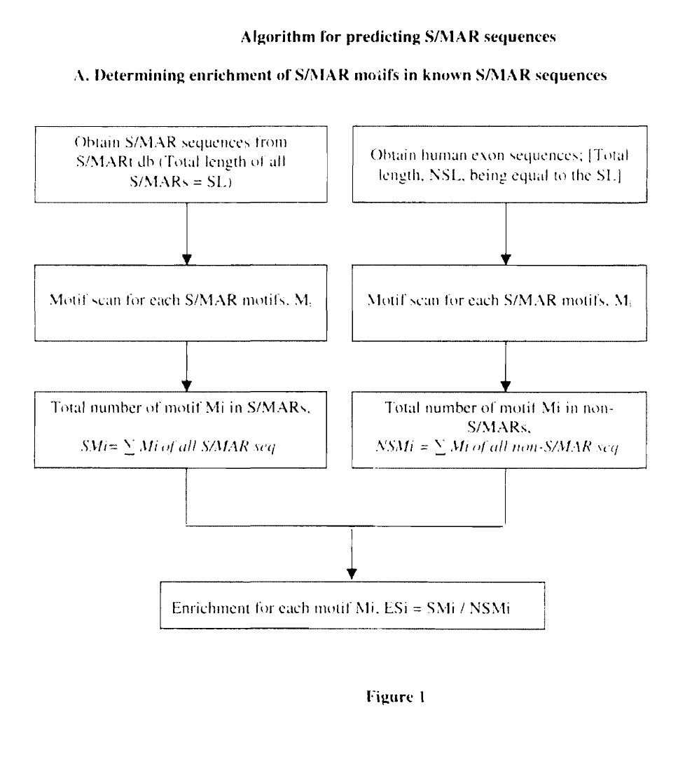

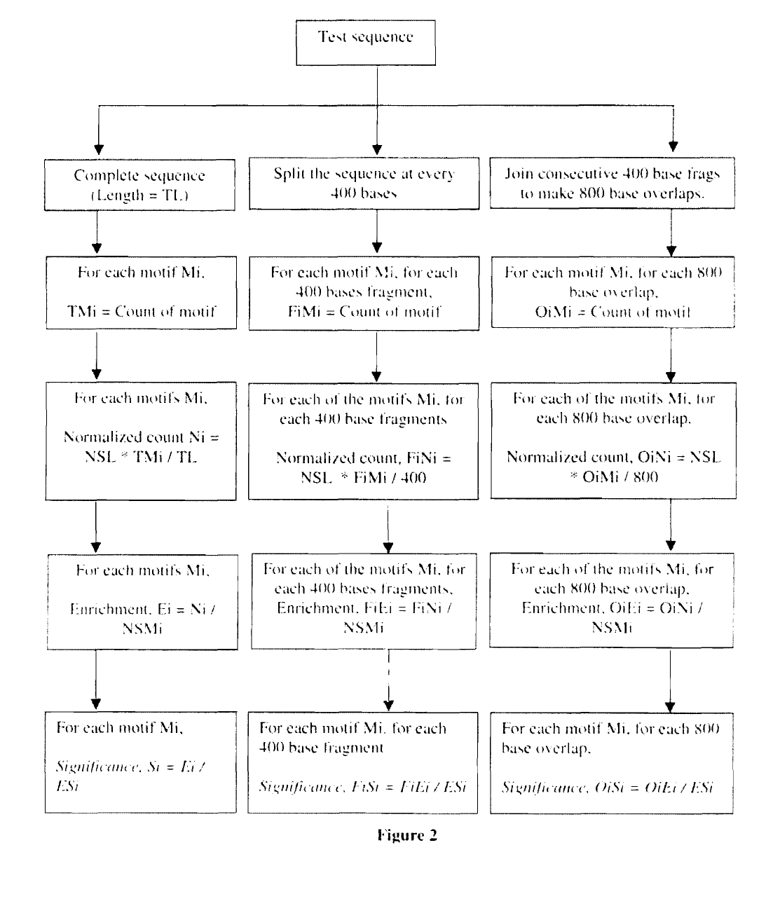

Algorithm for predicting S/MAR sequences is explained in FIGS. 1 and 2.

All sequences and fragments and overlaps with a significance value >0.9, is a potential S/MAR sequence.

Algorithm Explained

Identifying Potential S/MAR Sequences and S/MAR Regions

A. Obtain Knowledge from Known S/MAR Sequences
Get experimentally proved vertebrate S/MAR sequences. (Take from SMARt db)
Calculate the total length of the S/MAR sequences.
Calculate the occurrence of each of the motifs in each of the sequence and tabulate them.
For a particular motif, get the total number of times it is appearing in all the sequences.

Lets for example, say that the S/MAR1, S/MAR2 S/MAR3, S/MAR4 and S/MAR5 are known S/MAR sequences with the total length 10 KB. And the motifs 1, 2, 3 and 4 in them are as given in Table 2.

TABLE 2

| Seq | Motif 1 | Motif 2 | Motif 3 | Motif 4 |
|---|---|---|---|---|
| S/MAR1 | 3 | 6 | 3 | 1 |
| S/MAR2 | 5 | 2 | 6 | 4 |
| S/MAR3 | 1 | 0 | 3 | 2 |
| S/MAR4 | 8 | 4 | 3 | 0 |
| S/MAR5 | 4 | 3 | 8 | 2 |
| Total | 21 | 15 | 23 | 9 |

B. Obtain Knowledge from Non-S/MAR Sequences
Get exon sequences such that the total length of the entire exons equal the total length of MARs considered above.
Calculate the occurrence of each of the motifs in each of the sequence and tabulate them.
For a particular motif, get the total number of times it is appearing in all the sequences.

Lets for example, say that the Non-S/MAR1, Non-S/MAR2, Non-S/MAR3, Non-S/MAR4 and Non-S/MARS are exon sequences with the total length 10 KB. And the motifs 1, 2, 3 and 4 in them are as given in Table 3.

TABLE 3

| Seq | Motif 1 | Motif 2 | Motif 3 | Motif 4 |
|---|---|---|---|---|
| Non-S/MAR1 | 1 | 0 | 2 | 1 |
| Non-S/MAR2 | 0 | 1 | 3 | 0 |
| Non-S/MAR3 | 1 | 2 | 1 | 1 |
| Non-S/MAR4 | 2 | 0 | 0 | 0 |
| Non-S/MAR5 | 2 | 1 | 3 | 0 |
| Total | 6 | 4 | 8 | 2 |

Lets say that the length of sequences considered for S/MAR and non-S/MAR are 10,000 bp long. Since the length of sequences considered is the same, dividing the number of times a motif is appearing in S/MAR by number of times the same motif is appearing in non-S/MAR, gives the number of times a motif is enriched in S/MAR sequences than non-S/MAR sequences.

So in the above, the number of times each of the motif is enriched in MARs when compared to non-MARs are,

Motif 1=21/6=3.5

Motif 2=15/4=3.75

Motif 3=23/8=2.875

Motif 4=9/2=4.5

So, motifs 1, 2, 3 and 4 are likely to be represented 3.5, 3.75, 2.875 and 4.5 times more likely to be present in S/MAR sequences than non-MAR sequences. So any sequence that contains any of the motifs at or above these thresholds is a potential candidate to be a S/MAR sequence.

C. Finding Potential S/MAR Sequences

We take our sequences and calculate the occurrence of each of the motifs in our sequences. For each sequence, we calculate the motif occurrences by three ways:
Complete sequence
Split by 400 bases
Join consecutive 400 base sequences to make overlapping regions of 800 bases.

The number of times that the motifs are appearing will be normalized for 10 kb to check their significance of the complete sequence and the different segments. For example, lets take a 2.0 KB sequence. This sequence is analyzed as,

Complete Sequence:

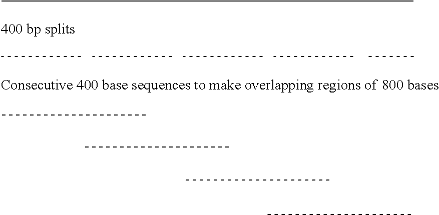

Calculate the occurrence of each of the motifs in the complete sequence and the various splits (Table 4)

TABLE 4

| Sequence | Motif 1 | Motif 2 | Motif 3 | Motif 4 |
|---|---|---|---|---|
| Complete | 6 | 2 | 3 | 4 |
| 400 bp splits | | | | |
| 1^{st} part | 1 | 0 | 0 | 1 |
| 2^{nd} part | 0 | 0 | 1 | 0 |
| 3^{rd} part | 2 | 1 | 1 | 0 |
| 4^{th} part | 1 | 0 | 0 | 1 |
| 5^{th} part | 2 | 1 | 1 | 2 |
| Overlapping | | | | |
| segments | | | | |
| 1^{st} overlap | 1 | 0 | 1 | 1 |
| 2^{nd} overlap | 2 | 1 | 2 | 0 |
| 3^{rd} overlap | 3 | 1 | 1 | 1 |
| 4^{th} overlap | 3 | 2 | 1 | 3 |

Motif Enrichment in the Complete Sequence

Motif 1 is appearing 6 times in 2 kb. Therefore for a 10 kb length, it will appear 30 times. So the enrichment of the number of motif 1 in this sequence when compared to non-MAR sequence is

30/6=5 [Note: 6 is the number of times motif 1 is appearing in non-S/MAR sequence for 10 KB]

Likewise, motifs 2, 3 and 4 appear with an enrichment of 2.5, 1.875 and 10 respectively.

Note: The base enrichment for motifs 1-4 calculated from known S/MAR sequences is 3.5, 3.75, 2.875 and 4.5 times respectively.

Hence, here motifs 1 and 4 are enriched more than base.

Motif Enrichment in 400 Base Region

Now, to find a region in this complete sequence that can be S/MAR, we will calculate the enrichment of each the motifs in the 400 bp fragments and the 800 bp overlaps.

For the first 400 bp fragment, motif 1 is appearing 1 time. So when it is normalized to 10 KB, it will contain

10000/400*1=25 times.

Likewise, the 1^{st} 400 bp part will contain the motifs 2, 3 and 4, 0, 0 and 25 times respectively.

The complete table for all the 400 bp fragments is given in Table 5.

TABLE 5

| Fragment | Motif 1 | Motif 2 | Motif 3 | Motif 4 |
|---|---|---|---|---|
| 1^{st} part | 25 | 0 | 0 | 25 |
| 2^{nd} part | 0 | 0 | 25 | 0 |
| 3^{rd} part | 50 | 25 | 25 | 0 |
| 4^{th} part | 25 | 0 | 0 | 25 |
| 5^{th} part | 50 | 25 | 25 | 50 |

For a 10 KB non-MAR fragment has 6, 4, 8 and 2 times of motifs 1, 2, 3 and 4 respectively (Table 6).

TABLE 6

| | Motif 1 | Motif 2 | Motif 3 | Motif 4 |
|---|---|---|---|---|
| Fragment | enrichment | enrichment | enrichment | enrichment |
| 1^{st} part | 4.16 | 0 | 0 | 12.5 |
| 2^{nd} part | 0 | 0 | 3.125 | 0 |
| 3^{rd} part | 8.3 | 6.25 | 3.125 | 0 |
| 4^{th} part | 4.16 | 0 | 0 | 12.5 |
| 5^{th} part | 8.3 | 6.25 | 3.125 | 25 |

The base enrichment for motifs 1-4 calculated from known sequences is 3.5, 3.75, 2.875 and 4.5 times respectively. From the above table, 5^{th} part has the most potential to be a S/MAR segment followed by 3^{rd} part.

Motif Enrichment in 800 bp Overlap Region

For the first 800 bp fragment, motif 1 is appearing 1 time. So when it is normalized to 10 KB, it will contain

10000/800*1=12.5 times

Likewise, the 1^{st} 400 bp part will contain the motifs 2, 3 and 4, 0, 12.5 and 12.5 times respectively.

The complete table for all the 800 bp overlaps is given in Table 7.

TABLE 7

| Fragment | Motif 1 | Motif 2 | Motif 3 | Motif 4 |
|---|---|---|---|---|
| 1^{st} overlap | 12.5 | 0 | 12.5 | 12.5 |
| 2^{nd} overlap | 25 | 12.5 | 25 | 0 |
| 3^{rd} overlap | 37.5 | 12.5 | 12.5 | 12.5 |
| 4^{th} overlap | 37.5 | 25 | 12.5 | 37.5 |

For a 10 KB non-MAR fragment has 6, 4, 8 and 2 times of motifs 1, 2, 3 and 4 respectively (Table 8).

TABLE 8

| | Motif 1 | Motif 2 | Motif 3 | Motif 4 |
|---|---|---|---|---|
| Fragment | enrichment | enrichment | enrichment | enrichment |
| 1^{st} overlap | 2.08 | 0 | 1.5625 | 6.25 |
| 2^{nd} overlap | 4.16 | 3.125 | 3.125 | 0 |
| 3^{rd} overlap | 6.25 | 3.125 | 1.5625 | 6.25 |
| 4^{th} overlap | 6.25 | 6.25 | 1.5625 | 18.75 |

The base enrichment for motifs 1-4 calculated from known sequences is 3.5, 3.75, 2.875 and 4.5 times respectively.

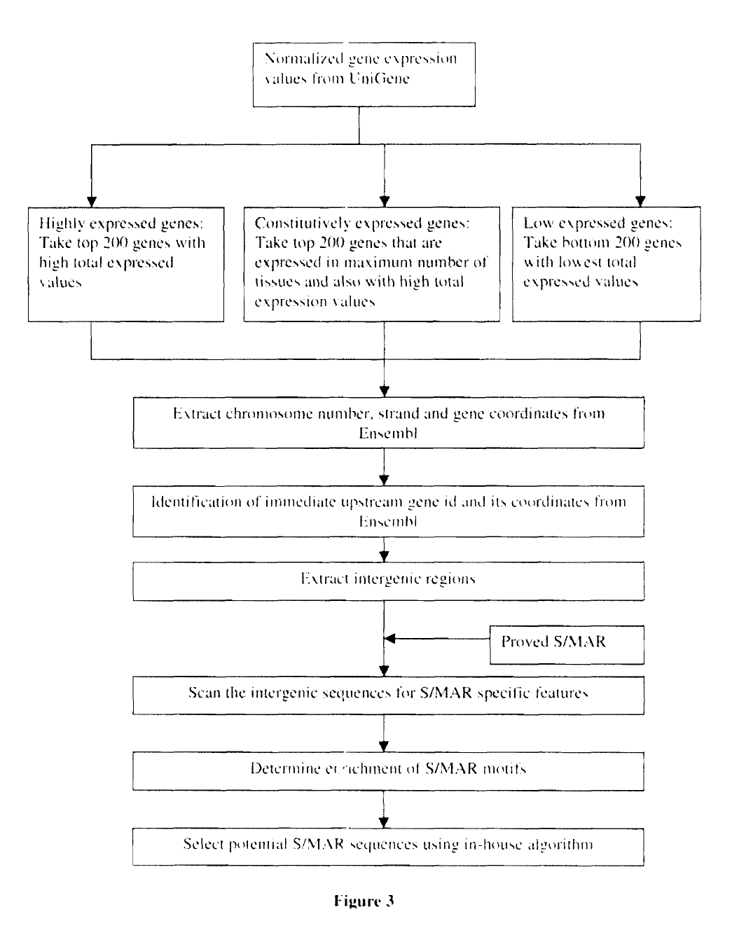

From the above table, 4^{th} 800 overlap, which is made up of 4^{th} and 5^{th} 400 bp fragments is the most enriched for all the motifs except for motif 3. Since the 5^{th} 400 bp fragment is enriched in all the motifs and since the enrichment of motif 3 is reduced in the 4^{th} overlap after combining the 5^{th} 400 bp fragment with the 4^{th} 400 bp fragment, it shows that the 5^{th} 400 bp fragment is the most S/MAR potential region. The second best region could be the 3^{rd} 800 bp overlap, which is a combination of 3^{rd} and 4^{th} 400 bp regions, which is also proved by the enrichment of motifs in the 3^{rd} 400 bp fragment. S/MAR Workflow is represented in FIG. 3.

Methodology

A. Database

For each gene, for each tissue type, the transcript per million copies (TPM) was calculated from the given expression values. The number of tissues in which the gene is expressed and the total expression value and the average expression value were calculated. A database of this was created. The database structure is as follows (Table 9)

TABLE 9

| Field | Type |
|---|---|
| Hs_no | varchar(10) |
| 2-46 TPM expression values in | int(10) |
| different tissue types | |
| exp_tissue_count | int(10) |
| total_exp | int(10) |
| avg_exp | int(10) |

B. Selecting Genes Based on Expression Values

Highly expressed genes: Genes were sorted based on the normalized UniGene total expression and the top 200 genes with the highest expression values were selected.

Constitutively expressed genes: Genes were sorted based on the number of tissues in which they are expressed and then on the normalized UniGene total expression. 200 genes with are expressed in the highest number of tissues and also with the highest expression values were selected.

Low expressed genes: Genes were sorted based on the normalized UniGene total expression and the bottom 200 genes with the lowest expression values were selected.

C. Intergenic Sequence Retrieval

S/MARs are found in non-coding sites. So, we extracted the intergenic region corresponding to all the gene obtained from UniGene and analyzed them for S/MAR specific features.

For a particular gene, the chromosome number, strand and gene coordinates were extracted from Ensembl 48. Based on the gene coordinates and gene strand, the coordinates for the immediate upstream gene was then retrieved. Based on the above two information, the intergenic region sequence was extracted.

D. Analysis of intergenic sequences for S/MAR specific features
16 S/MAR specific sequence motifs were collected from literature survey.
The proved S/MAR sequences and the intergenic sequences from high, constitutive and low expressed genes are scanned for the presence of these motifs. The A/T percentage is also calculated.
Enrichment of the S/MAR motifs are identified from proved S/MAR sequences
Selection of putative S/MAR sequences using the inhouse algorithm

Analysis

The Data Set

The sequences analyzed are

1. S/MAR sequences of Human, mouse, rat and chicken. The total length of sequences from S/MARt DB is 160 KB

2. Two sets of data based on expression level of genes from UniGene
a. Constitutively expressed gene set: Genes that are expressed in all the tissues. Order them by the decreasing order of the total expression level. Take the top 500. Get the corresponding ENSG ID. Corresponding ENSG IDs were obtained for 279 genes. Get the upstream intergenic region of these genes.
b. Low expressed gene set: Order the UniGene by the decreasing order of the expression level. Take the bottom 10000 genes. Get the corresponding ENSG IDs. Corresponding ENSG IDs were obtained for 212 genes. Get the upstream intergenic region of these genes.
   The total intergenic length for the constitutively and low expressed genes is 15090 and 16296 KB respectively.

3. 160 KB of exon sequences from Human Chr 22 (Since the total S/MAR sequences available from S/MARt DB was only 160 KB, only 160 KB of exons were taken)

The Analysis

The above sequences were scanned for 16 S/MAR motifs identified from literature. These sequences were scanned for the patterns only directly. They were NOT searched by the reverse of the S/MAR motif patterns.

Difference in motif concentration among S/MARt DB seq., intergenic region of constitutive and low expressed genes and exon sequences

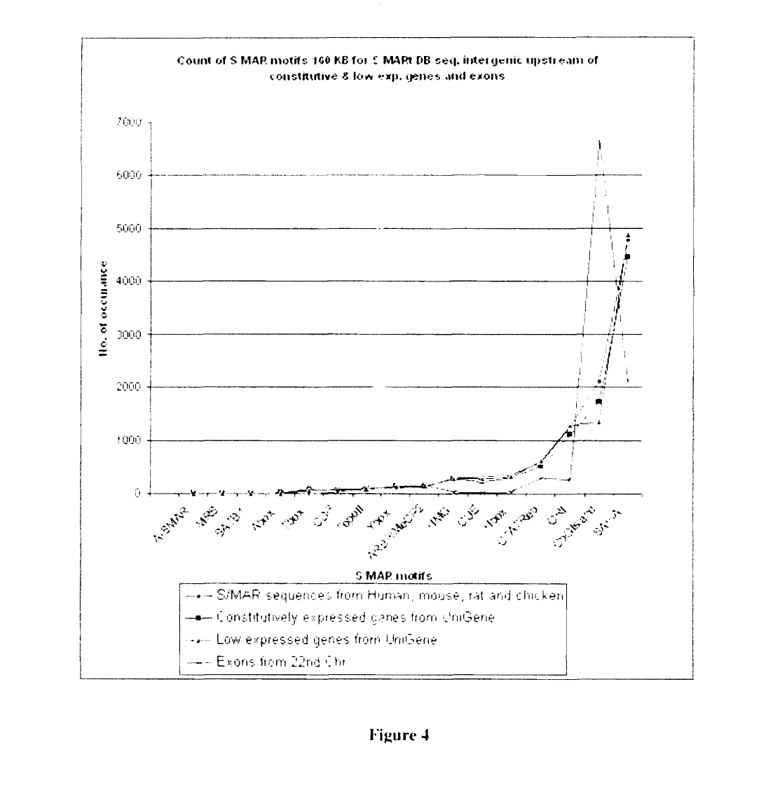

The motif counts for the four sets of sequences were calculated for 160 KB sequence was calculated and have been plotted (FIG. 4).

Two Points that are Clear from the Graph is that
a. The counts of motifs for all the motifs are low for exon sequences except for CpG islands
b. The counts of motifs for all the motifs are similar for sequences from S/MARt DB and constitutive and low expressed genes.

Motif Counts are Dependent on Length of the Intergenic Sequence

On sorting the motif counts for constitutive and low expressed genes, the counts of motifs are highly correlated with the sequence length for both the constitutive and low expressed genes.

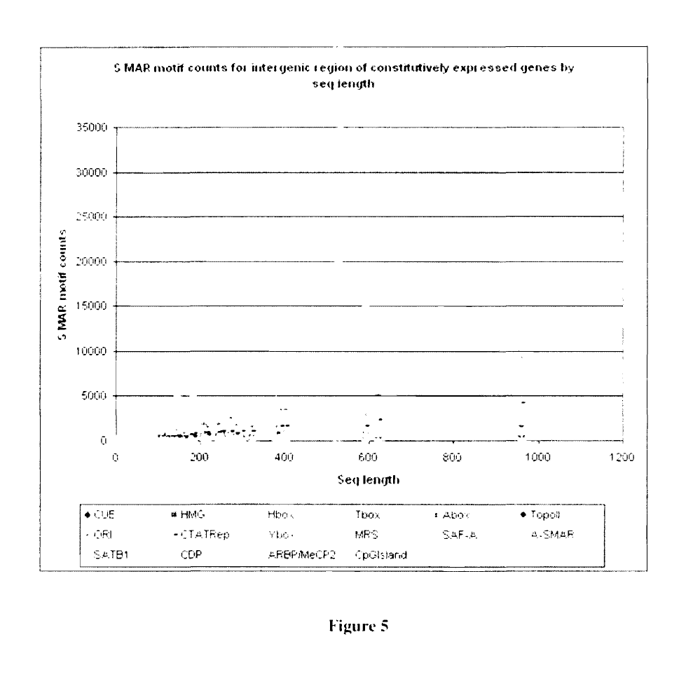

Graphs of S/MAR motif counts for constitutively and low expressed genes by length of the sequences (FIG. 5, 6)

Average Concentration of S/MAR Motifs per KB

Since the sequences vary in length, to normalize the S/MAR counts for the sequence length, we took the average count of S/MAR motifs per KB of sequence for each of the sequences to see if there is a higher concentration of S/MAR motifs in constitutively expressed genes than low expressed genes. From the graph below, both the constitutive and low expressed genes have the same average concentration of S/MAR motifs per KB.

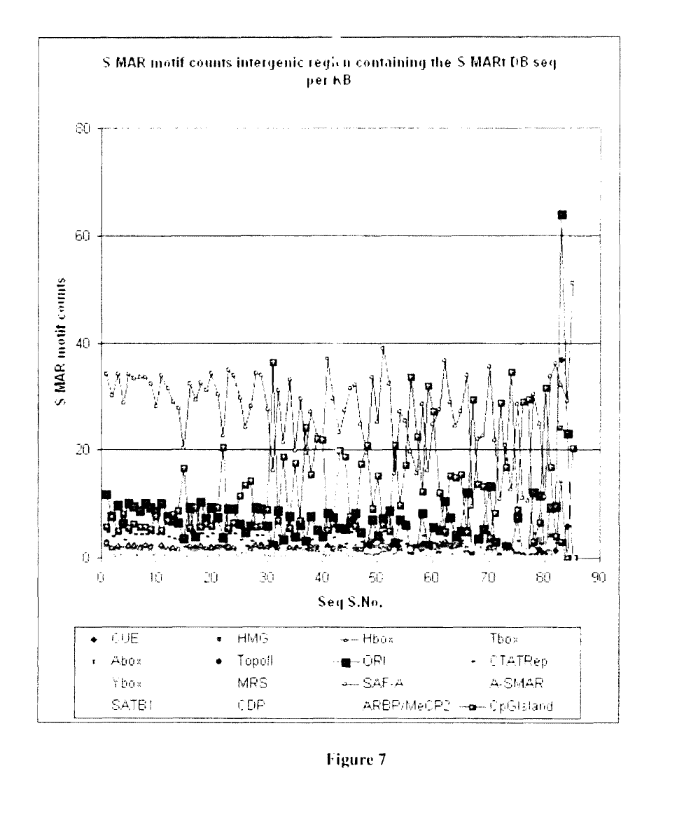

Graphs of average S/MAR motif counts per KB for the complete intergenic region containing the S/MARt DB sequence, upstream intergenic region of constitutively and low expressed genes by length of the sequences (FIG. 7, 8, 9, 10)

Note: The intergenic regions of constitutively and low expressed genes are arranged by the decreasing total expression values of the downstream gene.

Discussion and Directions for Analysis

1. Based on the Count of the Motifs

The sequences from S/MARt DB are having the highest number of positive S/MAR motifs. The intergenic regions of constitutive and low expressed genes motif counts are close to S/MARt DB sequences. Exon sequences have the lowest count of positive S/MAR motifs. This is as expected.

However, the intergenic regions upstream of low expressed genes are having higher number of positive S/MAR motifs than that for constitutively expressed genes.

This could happen for three reasons
1. If the gene selection for constitutive and low expressed genes are not according to the biological expression levels.
2. The high expression of some of the constitutive expressed genes is due to some other factors other than S/MAR sequences
3. The low expression of low expressed genes are repressed by factors that we do not know even though they have S/MAR motifs in them

Testing Reason 1

Assumption: If we assume that S/MAR sequences increase the expression levels of the genes downstream of it, we would expect genes downstream of proved S/MARt DB S/MAR sequences have high expression levels.

Since the constitutive and low expressed genes were taken from UniGene database based on the total expression value, we need to validate the expression values in UniGene.

Action

To test the above assumption,
For each of the S/MARt DB Human S/MAR sequence, get the gene downstream of it.
Get the expression value of that gene in UniGene

What can be Understood
Whether all genes downstream of S/MARs are highly expressed. If this is the case, then the assumption is correct.
Whether low expressed genes have positive S/MAR sequences upstream of them. Then there has to be an explanation for the low expression though they have S/MARs upstream of them.

2. Tissue Specificity of Motifs

In the analysis of the motifs there are low expressed genes that have equal or even more counts for positive S/MAR motifs than constitutive expressed genes. The constitutive and low expressed genes were selected based on the total expression of that gene in all the tissues and also the average expression of that gene.

Assumption:

Low expressed genes could be that are expressed in few tissues and blocked in others. There could be few motifs that influence the expression of a gene in specific tissues.

Hence if there is a gene that is only expressed in one or two tissue but they are enriched in motifs that help in that gene's expression in that tissue, then those motifs will be present in more counts in low expressed genes as well. So, the equality of the motif counts in constitutive and low expressed genes could be because of this tissue specificity.

Action:

To check the assumption, we will select two sets of genes,
Genes that are expressed in only one specific tissue type. E.g. Genes expressed only in adipose tissue
All genes that are expressed in a specific tissue type, regardless of whether they are expressed in other tissue types.

Evidences for the Tissue Specificity of S/MAR Sequences: References
1. Mathematical model to predict regions of chromatin attachment to the nuclear matrix, Nucleic Acids Research, 1997, Vol. 25, No. 7 1419-1425

Matrix attachment regions have been categorized as constitutive (permanent) or facultative (cell-type specific) (2). The constitutive MARs occur in all types of cells irrespective of the tissue in which they are found. In contrast, the presence of a facultative MAR is tissue specific and its use is governed by that tissue. MARs have been experimentally defined for several gene loci, including the chicken lysozyme gene (5), human interferon-b gene (6), human b-globin gene (7), chicken a-globin gene (8), p53 (9) and the human protamine gene cluster (10).
2. Nucleic Acids Research, 1996, Vol. 24, No. 8 1443-1452

The chicken lysozyme locus is regulated by a set of well characterized cis-regulatory elements each responsible for a distinct subaspect of tissue specificity of expression (27-33).
3. Transcriptional Activation by a Matrix Associating Region-binding Protein, The Journal of Biological Chemistry Vol. 276, No. 24, Issue of June 15, pp. 21325-21330, 2001

Transgenic studies have demonstrated that high level tissue-specific expression is only seen when the core is present in context of the MARs (8). This effect requires the core, because MARs alone could not produce high level expression. Although the MARs had previously been implicated in negative regulation of the Ig locus in non-B cells (4, 9-12), this was the first demonstration that the MARs were required for proper expression in B cells.
4. Identification and analysis of a matrix-attachment region 5′ of the rat glutamate-dehydrogenase-encoding gene, Eur. J. Biochem. 215, 777-785 (1993)

However, in these latter experiments, the level of expression was not copy-number dependent. This most likely results from the absence of MAR sequences at both sides of every whey acidic protein gene, since transgenic mice carrying the complete chicken lysozyme gene locus, including its 5′-located and 3′-located MAR sequences, showed not only accurate tissue specific, but also copy-number-dependent expression of the transgene [14]. These results suggest that MAR sequences can indeed establish independently regulated genetic domains.
5. Analysis of the chromatin domain organisation around the plastocyanin gene reveals an MAR-specific sequence element in Arabidopsis thaliana, Nucleic Acids Research, 1997, Vol. 25, No. 19

The evolutionary conserved nature of S/MARs suggests that S/MAR binding proteins must be commonly and ubiquitously expressed. This is the case for SAF-A (70), but not for SatB1 and Bright. These latter proteins are tissue specific (68,69). We find this MRS only in Arabidopsis S/MARs and not in S/MARs from other organisms, suggesting that the MRS is a binding site for an Arabidopsis-specific protein. The observation that SatB1, although specifically expressed in thymus, is able to bind to a large variety of other S/MARs would point to a widespread distribution of ARID proteins with similar but not identical binding sites.

3. Distance of a S/MAR Motifs from the Starting of a Gene

Assumption:

The distance of a motif from the starting of a gene might be important than the count of the number of times a motif appears in a sequence. It could be that S/MAR motifs are all clustered at a specific distance from the gene and there is a region in the intergenic sequences that have high concentration of S/MAR motifs.

But what is the cut off for the distance from the origin of gene?

For chicken lysozyme gene, the S/MAR motifs in the region between 8.5 to 11.5 KB upstream of the gene are the ones that influence the expression of the gene and not immediately upstream.

Action: Count of motifs in individual 1 KB segment

To see if there is a region in the intergenic sequences that has high concentration of S/MAR motifs,
Take an intergenic region.
Divide that sequence into 1 KB segments starting from the downstream gene side.
Get the count of S/MAR motifs for each of the 1 KB segment

We claim:

1) A method for identifying Scaffold/Matrix attachment region(S/MAR) sequence, said method comprising steps of:
a) generating a library of subset of genes based on higher and constitutive gene expression predicted from datasets derived from human autonomic gene expression library; and
b) assessing 5′ UTR intergenic sequences for the subsets to identify the MAR sequence.

2) The method as claimed in claim 1, wherein the intergenic sequence was retrieved within a defined region of the genome using Ensembl Slice.

3) The method as claimed in claim 1, wherein the MAR sequence is selected from a group comprising structural motifs, DNA-unwinding motif, replication initiator protein sites, homo-oligonucleotide repeats, hexanucleotides motifs, stretches of either T or A residues, SATB1 recognition sequence, kinked DNA, intrinsically curved DNA and motif TTTAAA.

4) The method as claimed in claim 1, wherein the MAR sequence was identified by assessing 5′ UTR intergenic region using perl program.

5) A Scaffold/Matrix attachment region (S/MAR) sequence[s] or its complementary sequence[s], variant[s] and fragment[s] thereof.

6) The MAR sequences as claimed in claim 5, wherein the MAR sequences are selected from a group comprising structural motifs, DNA-unwinding motif, replication initiator protein sites, homo-oligonucleotide repeats, hexanucleotides motifs, stretches of either T or A residues, SATB1 recognition sequence, kinked DNA, intrinsically curved DNA and motif TTTAAA.

7) The Scaffold/Matrix attachment region (S/MAR) sequence[s] or its complementary sequence[s], variant[s] and fragment[s] as claimed in claim 5, wherein said sequence[s] increase protein production through enhanced expression of genes.

8) The method and the scaffold/matrix attachment region (S/MAR) sequences as substantially herein described with accompanying examples and figures.


SEQUENCE LISTING (5 sequences)

SEQ ID NO: 1 - DNA, 800 bp, Homo sapiens
  gene (1)..(800)
tatataatatattatatattatattataatatatttttatataatatattataatatattatatttttatataatatattatattataatatatttttatataatatattatattataatatatttttatataatatattatatattataatataatatatttttatataatatattatatattataatataatatattttatatacaatgtttatgttatatattttatatacaatgtttatgttatatattttatatacaatgtttatgttatatattttatatacaatgtttatattatatataaatataaatatatataaatatatataatatataaatattatatataatatttatataatatatataaatatctataaatatttataatataatataaaatataatatatatttatatataatataatatataaatatatttaatatataaatatatttataatatgtaaataaatatatttatttatagaatatacttaatatatattaaatatataatataatataaatatataatatattataaatatatattatatataatacatattatatactatattatcaatatataatatattatataatacatattatataatatattatatataatatataataatataattattatatataatatataataatataattattatatataatatataataatataattattatatataatacataatatatattttatatattatatataatatatataatatataaaatacatatataagataatatattat

SEQ ID NO: 2 - DNA, 800 bp, Homo sapiens
  gene (1)..(800)
tcaaaactgatttactactgccataaatatattaaataatgaagcatatataattaaaaatacacaggaaattttaaaaatctttttgtgggaataacataacagaatatatcagaattcttgtgttcatatggcatggatctatagtagttctacaaactacaaacatgtttgcagcagcttatggatgaaagaaactcaatgacagtgttgcaaaattttacaagaatcccaaatatatattatatatataatatcatatattatatataatatatgatattatatattgtatatatattatatatgatatatatttttatataatatatattttatatattttatattttacatataatatatatttttatatattatatattttatatattatatcatatatataatatattttatatatattttatatacaatatataatgtattttatatatattttatatacaatatataatatattttctatatattttatatataatatataatatattttctatatattttatatataatatataatatttttatatatattttatatataaaatatattatatataatatattttatatataaaatatattatatataaaatattttatataatatatattaaatataatatatataatatataaaatatatatattatatataatataatatataaaatatatatattatatataatatattatataaaatataaatatataatatattatataaaatatatatattatatataatatattatataaaatatatataa

SEQ ID NO: 3 - DNA, 800 bp, Homo sapiens
  gene (1)..(800)
ataatgtaatatataatatataatatattctataatatataatatattctataatgtaatatataatatataatatattctataatgtaatatataatatataatatattatagaatatattatataatacattatataatatattatataatacattatatgatatattatataatgtattatatgatatattatataatgtattatataatatattatataatgtattatataatatatcatataatgtattatataatatatcatataatgtattatatattatatattatataatgtattatatattatatattacataatgtaaaatataatatattatatatattacatatatatgtattatataatatatattatatattatattatgtaatatataatatataatatatattacatataaaatatataaaaatatatattatatataaaatataaaaatatatatattatatatataatatataatatataaaatatataaaatatatatgaaatatataaaatatataaaatatatattatatatataaaatatataaaatatattttatatataatatataaaatatatattatatatataaaatatataaaatatatattatatataatatatataatatataataaataaaatatataaaatatataaatatatattatatatttatatatattatatatattatatatattttatatataatatatattatatatcttatatattttatatattatatataaaatatatattatatatataatatat

SEQ ID NO: 4 - DNA, 800 bp, Homo sapiens
  gene (1)..(800)
tatatattacaatttgtataacctatacaatctttatatacaatatactttatatattatatataatatttatatacaatatactttatatattatataaatctttatatacaatatactttatatattatatataatctttatatattataaatatatagttttatatttataatatataaatatattacattttataactatatagttttatatttataatatataaatatattacattttataaaaatatatttttatatttataaaaccatataaatatatttttatatttatattaataaaactatataaatatattttatatttatattaataaaactatataaatatattttatatttatatcaataaaactatataaatatattttatatttatatcaataaaactatataaatatatttatatttatatcaataaacataaatatattttatttttatattaataaatataaatatattttatttttatattaataaatataaatatattttatttttataataaatataaatatattttatttttatattaaatataaatatattttatttttatattaaatataaatatattttatttttatattaaatataaatatattttatttttatattaataaatataaatgtattttatatttataatataaatgtattttatatttataatataaatgtattttatatttataatataaatgtattttatatttataatataaatgtattttatatttataatataaatgtattttatatttataatataaa

SEQ ID NO: 5 - DNA, 800 bp, Homo sapiens
  gene (1)..(800)
tataatatattatataaatactatatcaatataatatattatataagtactatattaatatagtatataaatactatattaatataatatagtatataaatactatattaatatattatataaatactatattaatataatatataaatactataataatatataaataatatattaatattatatataattatatattaaattacatataatatataaatatatattatataatatataaatatatattaaattatataaaatatatattaaattatatatataaaatatatattaaataatatataaaatatatattaaataatatataaaatatatattatgtaaaatatatattaaataatatataaaatatatattatataatatataaaacataaataatatataaaacatatattaaataatatataaaatataaaacatatattatataatatataaaatttatatattatatattatataaatatatttattatatatattatataaatatatatttatatataatataaatatatattatatattatataatatattaaaatatatataattaatataatatatattaataatatgtattatttaacccagtgtgtccaaaatattaccatttcaacatgcaatccatattttaaaattattgaagtattttacttttttttggtatgaagtcttcaaaatccagcatatactttacacttaaagtgtatctcagttttaagtgtttgagggtcccatgtggctggtggcccacttattgggaagcacag